(19) United States
(12) Patent Application Publication
(10) Pub. No.: US 2023/0398552 A1
(43) Pub. Date: Dec. 14, 2023

(54) DETECTION METHOD AND DETECTION APPARATUS

(71) Applicant: Panasonic Intellectual Property Management Co., Ltd., Osaka (JP)

(72) Inventors: SATOSHI ARIMOTO, Shiga (JP); TAKASHI KANNO, Osaka (JP); RUI HIRAOKA, Osaka (JP)

(21) Appl. No.: 18/457,404

(22) Filed: Aug. 29, 2023

Related U.S. Application Data
(63) Continuation of application No. PCT/JP2022/007211, filed on Feb. 22, 2022.

(30) Foreign Application Priority Data
Mar. 18, 2021 (JP) 2021-044777

(51) Int. Cl. B03C 5/00; G01N 15/14; G01N 33/68; B03C 5/02; G01N 27/447

(52) U.S. Cl. CPC B03C 5/005; G01N 15/1434; G01N 33/6854; B03C 5/022; G01N 27/44721

(57) ABSTRACT

In a detection method, first dielectric particles each capable of being bound to a first target substance and second dielectric particles each capable of being bound to a second target substance are caused to react with a sample that contains a first target substance and a second target substance, the second dielectric particles having a different dielectrophoretic property from the first dielectric particles, a first composite particle to which the first target substance is bound is separated from the other first dielectric particle, and a second composite particle to which the second target substance is bound is separated from the other second dielectric particle by causing dielectrophoresis in the sample after the reaction, and the first target substance contained in the separated first composite particle and the second target substance contained in the second composite particle are each detected.

### BACKGROUND

### 1. Technical Field

The present disclosure relates to a detection method and a detection apparatus for detecting target substances, such as a virus.

### 2. Description of the Related Art

Hitherto, optical detection methods and other methods have been provided that use the near field to detect minute target substances with high sensitivity. For example, in International Publication No. 2017/187744, a target substance is detected by measuring, for example, a reduction in an optical signal generated by applying a first magnetic field that moves a bound body formed by binding the target substance, a magnetic particle, and a fluorescent particle away from the surface of a detection plate where the near field is formed.

### SUMMARY

In an existing detection method as in International Publication No. 2017/187744 or the like, one target substance is treated as a target. For example, in a case where target substances are present, it is difficult to detect each of these target substances appropriately.

One non-limiting and exemplary embodiment provides a detection method and the like with which target substances can each be appropriately detected.

In one general aspect, the techniques disclosed here feature a detection method. The detection method includes causing first dielectric particles and second dielectric particles to react with a sample that contains a first target substance and a second target substance, the first dielectric particles being modified with first substances having a property of specifically binding to the first target substance, the second dielectric particles being modified with second substances having a property of specifically binding to the second target substance different from the first target substance and having a different dielectrophoretic property from the first dielectric particles, by causing dielectrophoresis in the sample after the reaction, separating a first composite particle that is a first dielectric particle to which the first target substance is bound among the first dielectric particles from the other first dielectric particle, and separating a second composite particle that is a second dielectric particle to which the second target substance is bound among the second dielectric particles from the other second dielectric particle, and detecting each of the first target substance and the second target substance, the first target substance being contained in the separated first composite particle, the second target substance being contained in the separated second composite particle.

A detection method or the like according to an aspect of the present disclosure enables target substances to be individually appropriately detected.

It should be noted that these general or specific embodiments may be implemented as a system, a method, an apparatus, an integrated circuit, a computer program, a computer readable recording medium, or any selective combination thereof. Examples of the computer readable recording medium include a nonvolatile recording medium such as a compact disc read-only memory (CD-ROM).

Additional benefits and advantages of the disclosed embodiments will become apparent from the specification and drawings. The benefits and/or advantages may be individually obtained by the various embodiments and features of the specification and drawings, which need not all be provided in order to obtain one or more of such benefits and/or advantages.

### BRIEF DESCRIPTION OF THE DRAWINGS

FIG. 1A is a perspective view of a schematic configuration of a detection apparatus according to an embodiment;

FIG. 1B is a diagram for describing particle species according to the embodiment;

FIG. 2 is a sectional view of a schematic configuration of the detection apparatus according to the embodiment;

FIG. 3 is a plan view of the configuration of a set of electrodes according to the embodiment;

FIG. 4A is a diagram illustrating an example of positive precipitation of dielectric particles in dielectrophoresis;

FIG. 4B is a diagram illustrating an example of negative precipitation of dielectric particles in dielectrophoresis;

FIG. 5 is a graph illustrating set frequencies of alternating current voltages according to the embodiment;

FIG. 6 is a diagram for describing a precipitation pattern for each particle species at each frequency in the embodiment; and

FIG. 7 is a flow chart illustrating a detection method according to the embodiment.

### DETAILED DESCRIPTIONS

In the following, an embodiment will be specifically described with reference to the drawings.

Note that examples in an embodiment to be described below are each intended to represent a general or specific example. Numerical values, shapes, materials, constituent elements, arrangement positions and connection forms of the constituent elements, steps, and the order of steps described in the following embodiment are examples, and are not intended to limit the scope of the claims. Each drawing is not necessarily precisely illustrated. In the individual drawings, substantially the same configurations are denoted by the same reference signs. Redundant description may be omitted or simplified.

In the following, terms indicating relationships between elements, such as parallel and perpendicular, and terms indicating the shapes of elements, such as rectangular, as well as numerical ranges are not meant to express strict meaning only, but also to include substantially equivalent ranges, for example, differences of a few percent.

In the following, detecting a target substance includes measuring the amount of the target substance (for example, the number of target substances or the concentration of the target substance) or the range of the target substance, in addition to finding the target substance and confirming the presence of the target substance.

### Embodiment

In the present embodiment, composite particles and unbound particles are separated by dielectrophoresis (DEP) in a liquid, and target substances contained in the separated composite particles are detected.

Dielectrophoresis is a phenomenon in which a force acts on a dielectric particle subjected to a non-uniform electric field. This force does not require the particle to be charged.

A target substance is a substance to be detected. Examples of the target substance include a molecule such as a pathogenic protein, a virus (such as a coat protein), or a bacterium (such as a polysaccharide). The target substance may also be referred to as a specimen or an object to be detected. In the present embodiment, a detection method will be described in which in a case where types of target substance are simultaneously present as target substances, each of the types of target substance is individually detected. In an embodiment to be described below, a case where three types of target substance are present is described; however, the number of types of target substance is not limited to three. A detection method according to the present disclosure is applicable to two types of target substance and is also applicable to four or more types of target substance.

In the following, a detection apparatus and a detection method that realize detection of target substances using dielectrophoresis will be specifically described with reference to the drawings.

### Configuration of Detection Apparatus

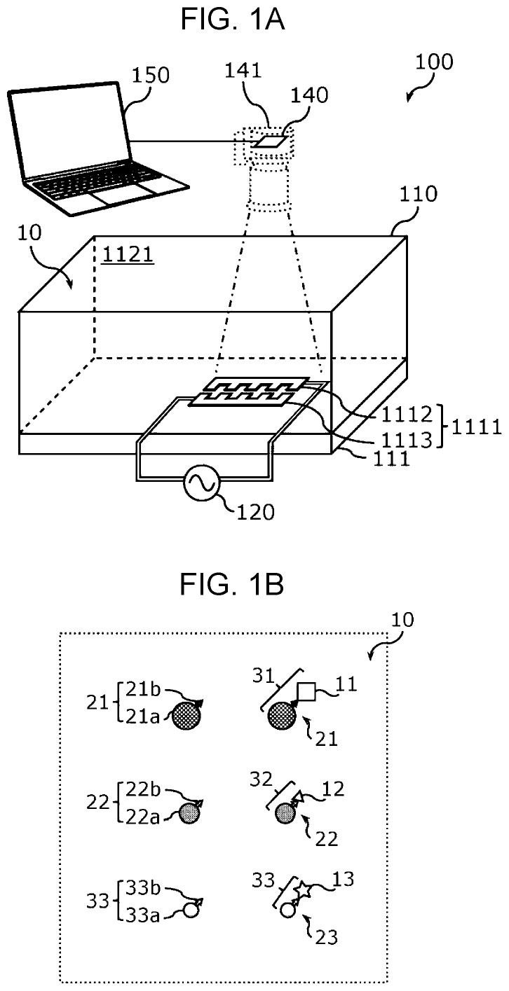

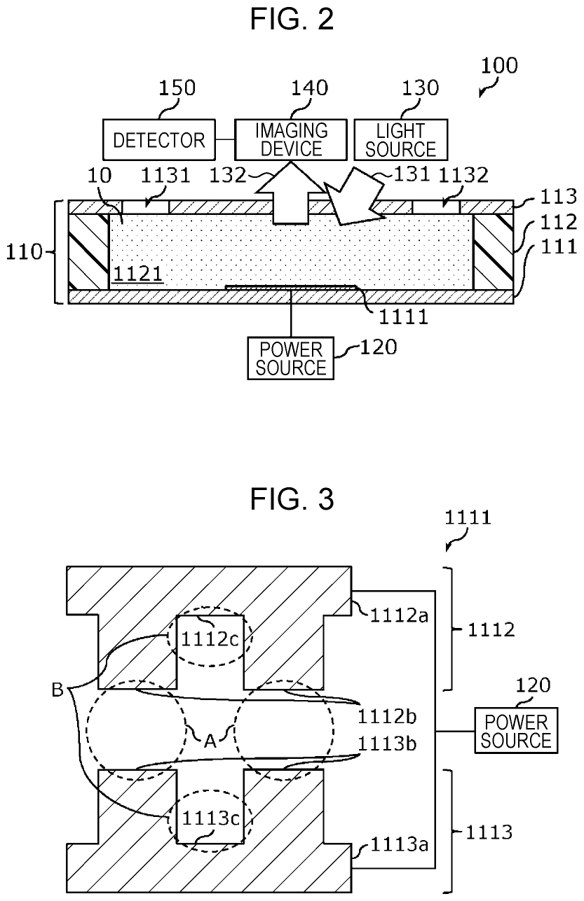

First, the configuration of a detection apparatus will be described with reference to FIGS. 1A, 1B, and 2. FIG. 1A is a perspective view of a schematic configuration of a detection apparatus according to an embodiment. FIG. 1B is a diagram for describing particle species according to the embodiment. FIG. 2 is a sectional view of a schematic configuration of the detection apparatus according to the embodiment. In FIG. 1A, in particular, a separator 110 is illustrated in outline so that the inside of the separator 110 can be seen through the portion of the separator excluding a first substrate 111. FIG. 1A is used to illustrate a relationship between the separator 110 and other constituent elements around the separator 110, and is not intended to limit, for example, the arrangement position, arrangement direction, or orientation of each constituent element when a detection apparatus 100 is used. FIG. 1B illustrates a sample 10 accommodated in a space 1121 in FIG. 1A, particle species (that is, dielectric particles) accommodated together with the sample 10 in this embodiment, and resulting particle species (that is, composite particles). FIG. 2 is a cross-sectional view of the separator 110 illustrated in FIG. 1A, the view being taken along a direction parallel to the paper surface. Note that the thickness of the configuration of part of the separator 110 illustrated in FIG. 2 is not illustrated in FIG. 1A.

As illustrated in FIGS. 1A and 2, the detection apparatus 100 includes the separator 110, a power source 120, a light source 130, an imaging device 140, and a detector 150.

The separator 110 is a container for accommodating the sample 10, which may contain target substances, and has a space 1121 inside. The space 1121 accommodates, together with the sample 10, types of dielectric particles corresponding to types of target substance in a respective manner. Thus, the space 1121 is also a site of a binding reaction in which types of target substance and types of dielectric particle bind to form types of composite particle. That is, in the present embodiment, the separator 110, which defines the space 1121, also functions as a reaction section. Note that the reaction section may be a container provided separately from the separator 110. In this case, the sample 10 after being reacted in another container is supplied into the container formed by the separator 110.

The separator 110 separates the composite particles, which are obtained by binding the target substances and the dielectric particles, from unbound particles, which are dielectric particles themselves, in the liquid (that is, in an outer liquid of the sample 10) by dielectrophoresis in the space 1121. In this case, the separator 110 positionally separates the composite particles from the unbound particles. In a case where the sample 10 contains unbound particles and target substances, the sample 10 further contains composite particles formed based on the target substances and the unbound particles. The sample 10 may be contaminated with a foreign substance.

In the present embodiment, in FIG. 1B, a first target substance 11 is illustrated as a rectangle, a second target substance 12 is illustrated as a triangle, and a third target substance 13 is illustrated as a star. The first target substance 11, the second target substance 12, and the third target substance 13 are contained in the sample 10. In the following, the first target substance 11, the second target substance 12, and the third target substance 13 may also be collectively referred to as and written as target substances. In the present embodiment, a first dielectric particle 21, a second dielectric particle 22, and a third dielectric particle 23 are contained together with the sample 10 so as to correspond to these target substances in a respective manner and are subjected to reaction.

The first dielectric particle 21 is created by modifying the surface of a first base material 21a with a first substance 21b, which has the property of specifically binding to the first target substance 11. Similarly, the second dielectric particle 22 is created by modifying the surface of a second base material 22a with a second substance 22b, which has the property of specifically binding to the second target substance 12. The third dielectric particle 23 is created by modifying the surface of a third base material 23a with a third substance 23b, which has the property of specifically binding to the third target substance 13. In this manner, each of the first base material 21a, the second base material 22a, and the third base material 23a is a base material portion of the corresponding dielectric particle excluding the substance having the property of specifically binding to the target substance. In the following, the first dielectric particle 21, the second dielectric particle 22, and the third dielectric particle 23 may be collectively referred to as and written as dielectric particles.

Note that the first base material 21a, the second base material 22a, and the third base material 23a have particle shapes and have different particle diameters from each other. For example, the first base material 21a, the second base material 22a, and the third base material 23a are sufficiently larger than the first substance 21b, the second substance 22b, and the third substance 23b corresponding thereto, respectively. As a result, the first base material 21a, the second base material 22a, and the third base material 23a have different dielectrophoretic properties. Thus, the first dielectric particle 21, the second dielectric particle 22, and the third dielectric particle 23 have different dielectrophoretic properties from each other.

Note that a particle diameter being different from another particle diameter means that the major peak of a particle size distribution that determines the particle diameter does not match the major peak of a particle size distribution that determines the other particle diameter. Thus, even in a case where the particle diameter of a particle species and that of another particle species are different from each other, some particles of the particle species and some particles of the other particle species may have the same particle diameter.

An example will be described in which base materials having different particle diameters are used as a condition for differentiating the dielectrophoretic properties of the first dielectric particle 21, the second dielectric particle 22, and the third dielectric particle 23; however, other configurations obtained by selecting, for example, particle materials (having different permittivities or conductivities from each other), surface charge states, or surface functional groups may be used as conditions. In this case, the first dielectric particle 21, the second dielectric particle 22, and the third dielectric particle 23 may be composed of base materials having the same particle diameter.

When the first base material 21a, the second base material 22a, and the third base material 23a are caused to have different particle diameters from each other with optically identifiable differences, the first base material 21a, the second base material 22a, and the third base material 23a can be identified from each other using, for example, a microscope. This effect will be further described below.

Dielectric particles are particles that can be polarized by an applied electric field. Dielectric particles may include, for example, a fluorescent substance. When light having a wavelength at which the fluorescent substance is excited is radiated from the light source 130 described below, the dielectric particles can be detected by detecting light in a fluorescence wavelength band.

In this case, the first base material 21a, the second base material 22a, and the third base material 23a have different spectroscopic properties from each other. Spectroscopic properties refer to, for example, fluorescence wavelength, excitation wavelength, transmittance and absorbance in a predetermined wavelength band, and spectral reflectance. Since the dielectric particles have different spectroscopic properties in this manner, the dielectric particles can be identified by type. This effect will be further described below. Note that each base material portion used in the dielectric particles is not limited to a base material that contains a fluorescent substance. For example, polystyrene particles, glass particles, or the like that do not contain a fluorescent substance may be used as base materials.

Each of the first substance 21b, the second substance 22b, and the third substance 23b described above is realized as an antibody that specifically binds to one corresponding type of target substance. For example, dielectric particles are formed by chemically bonding (modifying) the functional group on the surface of each base material and the stationary region of the corresponding antibody. Note that the first substance 21b, the second substance 22b, and the third substance 23b are not limited to antibodies, and can also be DNA aptamers, enzymes, or receptors, for example. In order to satisfy the property of specifically binding to a corresponding target substance, it is sufficient that at least a binding affinity for the corresponding target substance be higher than binding affinities for the other substances in the system for the sample 10. There may be no cross-reaction between the first substance 21b and the second target substance 12 or the third target substance 13. There may be no cross-reaction between the second substance 22b and the first target substance 11 or the third target substance 13. There may be no cross-reaction between the third substance 23b and the first target substance 11 or the second target substance 12.

A composite particle is a composite of one target substance and one dielectric particle corresponding to the target substance. That is, in the composite particle, the target substance and the dielectric particle are bound to each other with a substance having the property of specifically binding to the target substance interposed therebetween. In the present embodiment, the first target substance 11 and the first dielectric particle 21 form a first composite particle 31, the second target substance 12 and the second dielectric particle 22 form a second composite particle 32, and the third target substance 13 and the third dielectric particle 23 form a third composite particle 33.

An unbound particle is a dielectric particle with which a composite particle is not formed. That is, an unbound particle is a dielectric particle that is not bound to a target substance. An unbound particle is also called a free (F) component. In contrast, a dielectric particle of a composite particle, which corresponds to an unbound particle portion, is called a binding (B) component.

The internal configuration of the separator 110 is described below. As illustrated in FIG. 2, the separator 110 includes the first substrate 111, a spacer 112, and a second substrate 113.

The first substrate 111 is, for example, a sheet of glass or resin. The first substrate 111 has a top surface that defines the bottom of the space 1121, and a set of electrodes 1111, to which alternating current (AC) voltages are applied from the power source 120, is formed on the top surface. The set of electrodes 1111 includes a first electrode 1112 and a second electrode 1113 and can generate a non-uniform electric field (also called an electric field gradient) on the first substrate 111. That is, the set of electrodes 1111 is an example of an electric field gradient generator that generates (or forms) an electric field gradient. Note that details of the set of electrodes 1111 will be described below using FIG. 3.

The spacer 112 is disposed on the first substrate 111. A through hole corresponding to the shape of the space 1121 is formed in the spacer 112. In other words, the space 1121 is formed by the through hole sandwiched between the first substrate 111 and the second substrate 113. As described above, the sample 10, which may contain composite particles and unbound particles, is introduced into the space 1121. The spacer 112 is an outer wall surrounding the through hole and has an inner side surface that defines the space 1121. The spacer 112 is composed of a material such as a resin having high adhesion to the first substrate 111 and the second substrate 113, for example.

The second substrate 113 is, for example, a transparent sheet of glass or resin and is disposed on the spacer 112. For example, a polycarbonate substrate can be used as the second substrate 113. A supply hole 1131 and a discharge hole 1132, which communicate with the space 1121, are formed in the second substrate 113 such that the supply hole 1131 and the discharge hole 1132 penetrate through the second substrate 113. The sample 10 and dielectric particles are supplied into the space 1121 through the supply hole 1131 and are discharged from the space 1121 through the discharge hole 1132. Note that the separator 110 may be configured without provision of the second substrate 113. That is, the second substrate 113 is not an essential constituent element. For example, the space 1121 for the separator 110 to serve as a container is formed by the first substrate 111 and the spacer 112, which define the bottom surface and the inner side surface, respectively.

The power supply 120 is an AC power supply and applies an AC voltage to the set of electrodes 1111 of the first substrate 111. The power source 120 can be any power source that can supply AC voltage and is not limited to any particular power source. An AC voltage may be supplied from an external power source, in which case the power source 120 need not be included in the detection apparatus 100.

The light source 130 irradiates the sample 10 in the space 1121 with irradiation light 131. The irradiation light 131 is emitted to the sample 10 through the second substrate 113, which is transparent. Detection light 132 corresponding to the irradiation light 131 is generated from the sample 10, the dielectric particles contained in the sample 10 are detected by detecting the detection light 132. For example, as described above, in a case where a dielectric particle contains a fluorescent substance, the fluorescent substance is excited by being irradiated with excitation light as the irradiation light 131, and fluorescence emitted from the fluorescent substance is detected as the detection light 132.

As for the light source 130, known technologies can be used without any particular limitation. For example, a laser such as a semiconductor laser or a gas laser can be used as the light source 130. As for the wavelength of the irradiation light 131 with which irradiation is performed from the light source 130, a wavelength at which the interaction between the irradiation light 131 and a substance contained in a target substance is weak. For example, in a case where the target substance is a virus, the irradiation light 131 having a wavelength of 400 nm to 2000 nm is selected. As for the wavelength of the irradiation light 131, a wavelength that a semiconductor laser can use (for example, 600 nm to 850 nm) may be used.

Note that the light source 130 need not be included in the detection apparatus 100. For example, in a case where the size of a dielectric particle is large, observation can be performed using a combination of optical devices such as lenses, and luminescence phenomena such as fluorescence emission need not be used. That is, the dielectric particles need not contain a fluorescent substance, and the irradiation light 131 need not be radiated from the light source 130 in this case. The sun, a fluorescent light, and the like are used as the light source 130, and the dielectric particles can be detected using natural light with which irradiation is performed.

Examples of the imaging device 140 include a complementary metal-oxide-semiconductor (CMOS) image sensor and a charge-coupled device (CCD) image sensor. The imaging device 140 generates and outputs an image upon receiving the detection light 132 generated from the sample 10. The imaging device 140 is, for example, built in a camera 141 or the like and disposed horizontally along a plate surface of the first substrate 111, and images a portion corresponding to the set of electrodes 1111 through an optical element (not illustrated) such as a lens included in the camera 141. In this manner, the imaging device 140 is used to image composite particles separated from unbound particles by the separator 110 and detect the target substances contained in the composite particles.

In an example in which dielectric particles contains a fluorescent substance, the imaging device 140 images fluorescence emitted from the fluorescent substance contained in the dielectric particles. Note that the detection apparatus 100 may include a photodetector instead of the imaging device 140. In this case, it is sufficient that the photodetector detect the detection light 132 such as fluorescence from a region on the first substrate 111 where composite particles separated by dielectrophoresis gather. Note that in a case where a photodetector is used instead of the imaging device 140 in this manner, for each type of dielectric particle, the number of dielectric particles is estimated on the basis of the strength of the detection light 132 using a correlation between the strength of detection light and the number of dielectric particles in an analysis performed by the detector 150 to be described below. For each type of dielectric particle, a target substance bound to the dielectric particle may be detected from this estimated value.

The detection apparatus 100 may include at least one out of an optical lens and an optical filter at least between the light source 130 and the separator 110 or between the separator 110 and the imaging device 140. For example, a long pass filter that blocks the irradiation light 131 from the light source 130 and allows the detection light 132 to pass therethrough may be installed between the separator 110 and the imaging device 140.

The detector 150 acquires images output by the imaging device 140 and detects, on the basis of the images, dielectric particles contained in the sample 10. In particular, in the detection apparatus 100 according to the present embodiment, the number of composite particles and the number of unbound particles can be separately counted.

That is, the first dielectric particle 21 contained in the first composite particle 31 and a first dielectric particle 21 that is an unbound particle are distinguished from each other and are detected. The second dielectric particle 22 contained in the second composite particle 32 and a second dielectric particle 22 that is an unbound particle are distinguished from each other and are detected. The third dielectric particle 23 contained in the third composite particle 33 and a third dielectric particle 23 that is an unbound particle are distinguished from each other and are detected. In the present embodiment, the first composite particle 31, the second composite particle 32, and the third composite particle 33 can be distinguished from each other and detected. The number of composite particles corresponds to the number of target substances in accordance with, for example, a certain binding ratio. Thus, by detecting dielectric particles on the basis of the images, the detector 150 can detect each of the first target substance 11, the second target substance 12, and the third target substance 13 contained in composite particles in the sample 10.

For example, the detector 150 detects bright spots with different luminance values by comparing, using a pre-captured reference image that does not include a dielectric particle, an acquired image with the reference image. Specifically, it is sufficient that spots with higher luminance values in the acquired image than in the reference image be treated as bright spots in a case where emitted light is detected as the detection light 132, and spots with lower luminance values in the acquired image than in the reference image be treated as bright spots in a case where transmitted light and scattered light, for example, are detected as the detection light 132. In this manner, the detector 150 detects each composite particle in the sample 10 and detects a target substance corresponding to the composite particle.

The detector 150 is realized, for example, by executing a program for the above-described image analysis using a circuit such as a processor and a storage device such as a memory; however, the detector 150 may be realized by a dedicated circuit. For example, the detector 150 is built in a computer.

### Shape and Arrangement of Set of Electrodes on First Substrate

Next, the shape and arrangement of the set of electrodes 1111 on the first substrate 111 will be described with reference to FIG. 3. FIG. 3 is a plan view of the configuration of the set of electrodes according to the embodiment. In FIG. 3, the configuration of the set of electrodes 1111 when viewed in a plan view from the imaging device 140 side is illustrated. Note that, for the sake of simplicity, FIG. 3 illustrates a schematic diagram of part of the set of electrodes 1111.

As described above, the set of electrodes 1111 has a first electrode 1112 and a second electrode 1113 disposed on the first substrate 111. Each of the first electrode 1112 and the second electrode 1113 is electrically connected to the power source 120.

The first electrode 1112 includes a first base 1112a and two first projections 1112b. The first base 1112a extends in a first direction (a horizontal direction on the sheet of FIG. 3). The two first projections 1112b protrude from the first base 1112a in a second direction (a vertical direction on the sheet of FIG. 3) that intersects the first direction. A first recess 1112c is formed between the two first projections 1112b. The two first projections 1112b are disposed so as to face the second electrode 1113. That is, the first electrode 1112 includes the first projections 1112b, which protrude from the first base 1112a in a direction that intersects the first direction so as to project toward the second electrode 1113. Note that the first projections 1112b face second projections 1113b of the second electrode 1113. The two first projections 1112b and the first recess 1112c each have, for example, lengths of about 10 micrometers in the first direction and the second direction. Note that the sizes of the two first projections 1112b and the first recess 1112c are not limited to this.

The shape and size of the second electrode 1113 are substantially the same as those of the first electrode 1112. That is, the second electrode 1113 also includes a second base 1113a and two second projections 1113b. The second base 1113a extends in a first direction (a horizontal direction on the sheet of FIG. 3). The two second projections 1113b protrude from the second base 1113a in a second direction (a vertical direction on the sheet of FIG. 3) that intersects the first direction. A second recess 1113c is formed between the two second projections 1113b. The two second projections 1113b are disposed so as to face the first electrode 1112. That is, the second electrode 1113 includes the second projections 1113b, which protrude from the second base 1113a in the direction that intersects the first direction so as to project toward the first electrode 1112. Note that the second projections 1113b face the first projections 1112b of the first electrode 1112.

By applying AC voltages to electrodes such as the first electrode 1112 and the second electrode 1113, a non-uniform electric field is generated on the first substrate 111. The AC voltage applied to the first electrode 1112 and the AC voltage applied to the second electrode 1113 may be substantially equal to each other, or a phase difference may be set between the AC voltages. For example, 180 degrees can be used as the phase difference between the AC voltages to be applied.

Note that the position of the set of electrodes 1111 is not limited to somewhere on the first substrate 111. It is sufficient that the set of electrodes 1111 be disposed near the sample 10 in the space 1121. In this case, “near the sample 10” refers to a region where an electric field can be generated in the sample 10 by the AC voltages applied to the set of electrodes 1111. That is, the set of electrodes 1111 may be directly in contact with the sample 10 in the space 1121, or may form, from outside the space 1121, an electric field in a region including the sample 10.

### Distribution of Electric Field Strength on First Substrate

In the following, an electric field strength distribution of the non-uniform electric field generated on the first substrate 111 will be described with reference to FIG. 3.

As illustrated in FIG. 3, the non-uniform electric field creates, on the first substrate 111, first electric field regions A where the electric field strength is relatively high and second electric field regions B where the electric field strength is relatively low. The first electric field regions A have higher electric field strength than the second electric field regions B. Each first electric field region A is a region between a corresponding first projection 1112b and a corresponding second projection 1113b, which face each other.

The electric field strength depends on the distance between electrodes that generate the electric field. The electric field strength decreases as the distance between the electrodes increases, and increases as the distance between the electrodes decreases. The positions where ends of the first projections 1112b face ends of the second projections 1113b in the first direction are positions of the set of electrodes 1111 where the distances between the first electrodes 1112 and the second electrodes 1113 are shortest and the electric field strength is highest. The first electric field regions A are regions having certain areas including the positions where the distances between the first electrodes 1112 and the second electrodes 1113 are shortest as described above.

The second electric field regions B have lower electric field strength than the first electric field regions A and are formed in a region between the first recess 1112c and the second recess 1113c, which face each other. This region is at a position where the distance between the first electrode 1112 and the second electrode 1113 is longest. In particular, the closer to the bottom of the first recess 1112c or to the bottom of the second recess 1113c, the lower the electric field strength. The second electric field regions B are regions that include the bottoms of the first recess 1112c and the second recess 1113c where the electric field strength is especially low.

### Positive Precipitation and Negative Precipitation by Dielectrophoresis

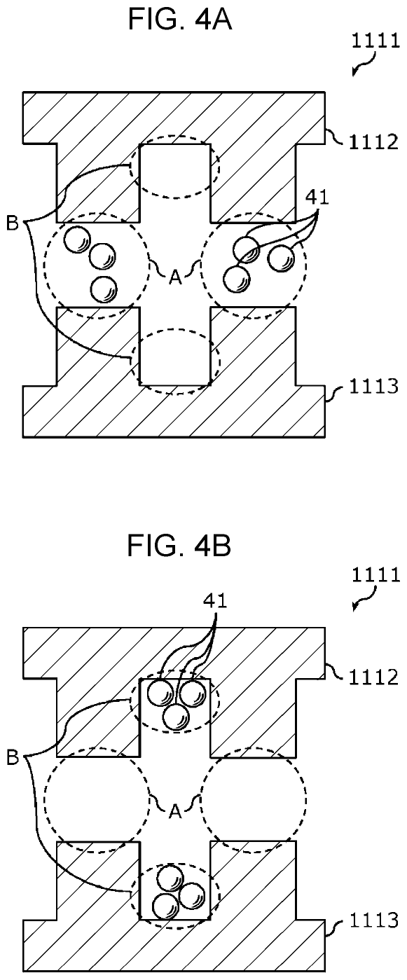

Positive precipitation and negative precipitation of dielectric particles for a case where the set of electrodes 1111, which is configured as above, is used will be described using FIGS. 4A and 4B. FIG. 4A is a diagram illustrating an example of positive precipitation of dielectric particles in dielectrophoresis. FIG. 4B is a diagram illustrating an example of negative precipitation of dielectric particles in dielectrophoresis. Note that, for the sake of simplicity, the behavior of dielectric particles 41 of one type in a case where the dielectric particles 41 are subjected to dielectrophoresis will be described using FIGS. 4A and 4B.

As illustrated in FIG. 4A, depending on, for example, the frequencies of AC voltages applied to the set of electrodes 1111 and ion species in an external liquid surrounding the dielectric particles 41, the dielectric particles 41 accumulate in the first electric field regions A, where the electric field strength is high. In this case, the behavior of the dielectric particles during dielectrophoresis (that is, whether positive precipitation occurs or negative precipitation occurs) is determined by the real part of the Clausius-Mossotti factor. In a case where the real part of the Clausius-Mossotti factor has a positive numerical value due to various conditions during the dielectrophoresis, positive precipitation of the dielectric particles 41 occurs in the first electric field regions A due to the effect of positive dielectrophoresis (pDEP) as in the drawing.

As illustrated in FIG. 4B, depending on, for example, the frequencies of AC voltages applied to the set of electrodes 1111 and ion species in the external liquid surrounding the dielectric particles 41, the dielectric particles 41 accumulate in the second electric field regions B, where the electric field strength is low. In this case, the behavior of the dielectric particles during dielectrophoresis (that is, whether positive precipitation occurs or negative precipitation occurs) is determined by the real part of the Clausius-Mossotti factor. In a case where the real part of the Clausius-Mossotti factor has a negative numerical value due to various conditions during the dielectrophoresis, negative precipitation of the dielectric particles 41 occurs in the second electric field regions B due to the effect of negative dielectrophoresis (nDEP) as in the drawing.

In the present embodiment, regarding dielectric particles of types (including composite particles), precipitation of the dielectric particles is changed from positive to negative one type at a time by changing the real part of the Clausius-Mossotti factor from positive to negative for each type of dielectric particle in a sequential manner, so that the respective dielectric particles are detected. As a result, target substances bound to the respective dielectric particles can be detected one type at a time. Note that, similarly to as in the above description, regarding dielectric particles of types (including composite particles), precipitation of the dielectric particles may be changed from negative to positive one type at a time by changing the real part of the Clausius-Mossotti factor from negative to positive for each type of dielectric particle in a sequential manner, so that the respective dielectric particles are detected.

In the present embodiment, the frequencies of the applied AC voltages are changed from low frequencies to high frequencies in order to change the real part of the Clausius-Mossotti factor from negative to positive. The external liquid surrounding the composite particles and dielectric particles may be changed in a sequential manner by titration or the like, or an interelectrode distance between the first electrode 1112 and the second electrode 1113 may be changed.

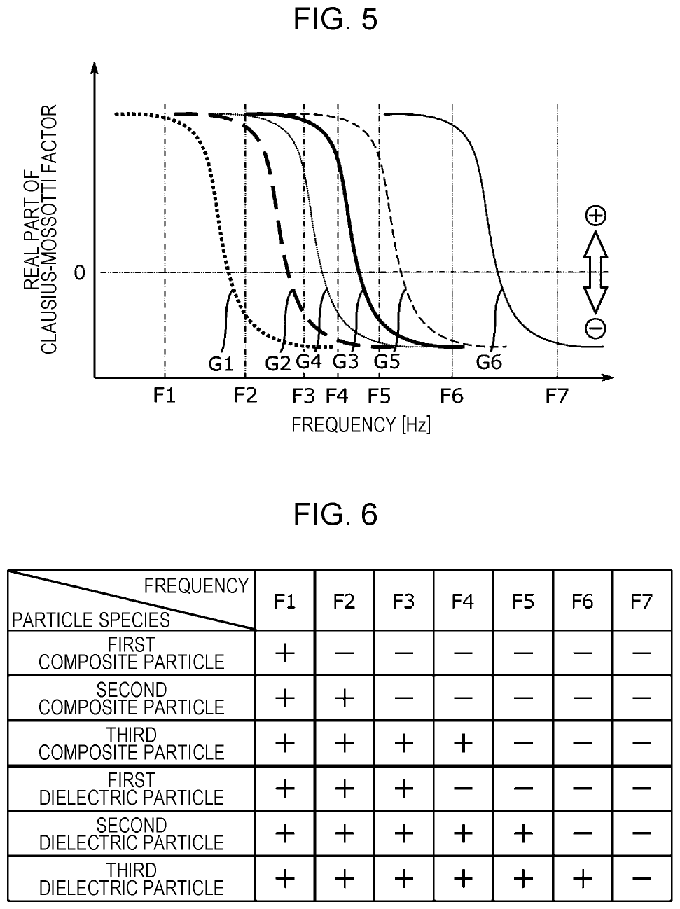

In the following, dielectric particle detection according to the present embodiment will be described using FIGS. 5 and 6. FIG. 5 is a graph illustrating set frequencies of AC voltages according to the embodiment. FIG. 6 is a diagram for describing a precipitation pattern for each particle species at each frequency in the embodiment.

In the graph illustrated in FIG. 5, the vertical axis represents the real part of the Clausius-Mossotti factor, and the horizontal axis represents the frequency of an AC voltage applied between the electrodes of the set of electrodes 1111. As described above, when the real part of the Clausius-Mossotti factor is positive, dielectric particles are affected by positive dielectrophoresis, and the dielectric particles move to a region having higher electric field strength. In contrast, when the real part of the Clausius-Mossotti factor is negative, dielectric particles are affected by negative dielectrophoresis, and the dielectric particles move to a region having lower electric field strength.

In FIG. 5, a graph G1 corresponding to the first composite particle 31 is illustrated by a thick dashed line, and a graph G4 corresponding to the first dielectric particle 21 that is unbound is illustrated by a thin dashed line. A graph G2 corresponding to the second composite particle 32 is illustrated by a thick long dashed line, and a graph G5 corresponding to the second dielectric particle 22 that is unbound is illustrated by a thin long dashed line. A graph G3 corresponding to the third composite particle 33 is illustrated by a thick solid line, and a graph G6 corresponding to the third dielectric particle 23 that is unbound is illustrated by a thin solid line. Note that, in FIG. 5, the position where the real part of the Clausius-Mossotti factor is 0 is illustrated by a dash-dot-dot line extending in a horizontal direction on the sheet.

As illustrated in the drawing, for each of the composite particles and dielectric particles, the real part of the Clausius-Mossotti factor is positive when the frequency of the applied AC voltage is in a low frequency band. When the frequency of the applied AC voltage is in a high frequency band, the real part of the Clausius-Mossotti factor is negative. Thus, by changing the frequency of the applied AC voltage from low frequencies to high frequencies, the real part of the Clausius-Mossotti factor changes from positive to negative. In this case, each of the composite particles and dielectric particles has a different frequency point at which the real part of the Clausius-Mossotti factor changes from positive to negative.

Thus, in this example, when the frequencies of the applied AC voltages are changed from low frequencies to high frequencies, precipitation of each of the composite particles and dielectric particles can be changed from positive to negative in a sequential manner.

FIG. 6 summarizes the behaviors of the composite particles and dielectric particles during dielectrophoresis at several frequency points. In FIG. 6, the first column illustrates types (particle species) of composite particle and types (particle species) of dielectric particle. The second to eighth columns illustrate the behaviors of the individual composite and dielectric particles at individual frequency points during dielectrophoresis. Note that, for combinations of particle species and frequency points in the second to eighth columns, plus signs indicate occurrence of positive precipitation, and minus signs indicate occurrence of negative precipitation. Note that the frequency points in FIG. 6 (a first frequency F1 to a seventh frequency F7) correspond to frequency points (a first frequency F1 to a seventh frequency F7) illustrated together with dash-dot-dot lines extending in the vertical direction on the sheet of FIG. 5.

As illustrated in FIGS. 5 and 6, for example, when an AC voltage at the first frequency F1 is applied, positive precipitation of each of the first composite particle 31, the second composite particle 32, the third composite particle 33, the first dielectric particle 21, the second dielectric particle 22, and the third dielectric particle 23 occurs.

Generally, the larger the size of the dielectric particle, the lower the frequency at which the real part of the Clausius-Mossotti factor changes from positive to negative. Thus, as in the present embodiment, by configuring the particle diameters of the first base material 21a, the second base material 22a, and the third base material 23a to be different from each other, it is possible to differentiate the frequency points at which the real part of the Clausius-Mossotti factor changes from positive to negative.

Note that, in a case where composite particles are formed, states such as surface charge may change, and the frequency points at which the real part of the Clausius-Mossotti factor changes from positive to negative may coincide with each other. In such a case, two types of composite or dielectric particles for which the real part of the Clausius-Mossotti factor has changed from positive to negative and whose precipitation has been changed from positive to negative at the same frequency point can each be detected by identifying a difference in particle diameter through optical observation using a microscope or the like.

Using the different spectroscopic properties of the first base material 21a, the second base material 22a, and the third base material 23a, two types of composite or dielectric particles whose precipitation has been changed from positive to negative at the same frequency point can also each be detected through identification made based on spectroscopic measurements. Even in cases where the real part of the Clausius-Mossotti factor for a foreign substance or the like that is not illustrated and the real part of the Clausius-Mossotti factor for any of the above particle species change from positive to negative at the same frequency point, it is effective to detect particle species using such spectroscopic properties.

Next, when an AC voltage at the second frequency F2 is applied, precipitation of the first composite particle 31 changes to negative. As a result, the first composite particle 31 can be separately detected, and thus the first target substance 11 contained in the first composite particle 31 can be detected. Next, when an AC voltage at the third frequency F3 is applied, precipitation of the second composite particle 32 further changes to negative. As a result, the second composite particle 32 can be separately detected, and thus the second target substance 12 contained in the second composite particle 32 can be detected.

Next, when an AC voltage at the fourth frequency F4 is applied, precipitation of the first dielectric particle 21 further changes to negative. As a result, the first dielectric particle 21 can be separately detected. Next, when an AC voltage at the fifth frequency F5 is applied, precipitation of the third composite particle 33 further changes to negative. As a result, the third composite particle 33 can be separately detected, and thus the third target substance 13 contained in the third composite particle 33 can be detected. In this manner, precipitation of the first dielectric particle 21 changes from positive to negative at a frequency lower than precipitation of the third composite particle 33 in the present embodiment. Because of this, the first dielectric particle 21 can be separately detected by applying the AC voltage at the fourth frequency F4, so that confusion between the third composite particle 33 and the first dielectric particle 21 can be suppressed.

Next, when an AC voltage at the sixth frequency F6 is applied, precipitation of the second dielectric particle 22 further changes to negative. As a result, the second dielectric particle 22 can be separately detected. Next, when an AC voltage at the seventh frequency F7 is applied, precipitation of the third dielectric particle 23 further changes to negative. As a result, the third dielectric particle 23 can be separately detected. In this way, precipitation of each of the particle species is sequentially changed from positive to negative by the AC voltage being applied at the first frequency F1 to the seventh frequency F7 in a time-division manner. In this manner, the first target substance 11, the second target substance 12, and the third target substance 13 can each be appropriately detected by the AC voltage being applied especially at the first frequency F1 to the fifth frequency F5 individually in the present embodiment.

Note that, in the above description, while precipitation of composite or dielectric particles that has been changed to negative is maintained as it is, precipitation of the next particle species is changed to negative. In contrast, composite or dielectric particles whose precipitation has been changed to negative may be collected in a sequential manner, and the number of types of particle species whose precipitation is changed to negative may be always set to less than or equal to one.

### Operation of Detection Apparatus

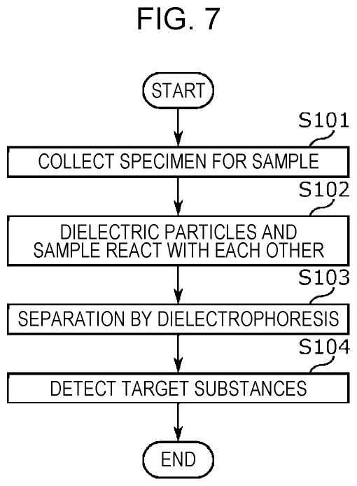

Next, a method for detecting target substances by operating the above-described detection apparatus 100 will be described with reference to FIG. 7. FIG. 7 is a flow chart illustrating a detection method according to the embodiment.

First, a specimen for detection to be used as the sample 10 is collected (S101). This is performed by the operation of a specimen collector, which is not illustrated. The specimen collector collects a specimen for detection by separating, from a fluid, a fraction that may contain target substances using, for example, a cyclone separator or a filter separator. Moreover, a known technology for separating a fraction that may contain target substances, such as electrostatic collection, can be selected and applied as desired for the specimen collector. Note that, although it depends on the configuration of the specimen collector, the fluid from which the fraction that may contain target substances is separated may be a gas or a liquid. In other words, the detection apparatus 100 can be used for any object by selecting a specimen collector corresponding to the properties of the fluid. In a case where a liquid fraction is obtained, the obtained fraction can be used as the sample 10 as it is. In a case where a gaseous fraction is obtained, the gaseous fraction is suspended in an aqueous solution such as phosphate-buffered saline and used as the sample 10.

Next, the sample 10 and the dielectric particles corresponding to the respective target substances are all accommodated in the space 1121 to cause a binding reaction (S102). As a result, in a case where target substances are contained in the sample 10, composite particles are formed. Next, the composite particles and dielectric particles that are unbound particles are separated from each other by dielectrophoresis in a liquid (an outer liquid of sample 10) (S103). Specifically, AC voltages are applied to the set of electrodes 1111 to generate a non-uniform electric field in the sample 10 on the first substrate 111. As a result, the composite and dielectric particles are affected by dielectrophoresis, resulting in positive or negative precipitation of each of the composite and dielectric particles.

For this phenomenon, the frequencies of the AC voltages are set to the first frequency F1 to the seventh frequency F7, which are described above, and are applied to the set of electrodes 1111 in a time-division manner. As a result of application of the AC voltages at the frequencies, dielectrophoresis can affect the composite and dielectric particles such that one type of particle species moves in a different direction. For example, when the frequency of an AC voltage is set to a frequency at which negative dielectrophoresis affects first composite particles 31, and positive dielectrophoresis affects the other particle species, the first composite particles 31 move to the second electric field regions B, where the electric field strength is relatively low, and the other particle species move to the first electric field regions A, where the electric field strength is relatively high.

Lastly, the target substances contained in the composite particles separated from the dielectric particles are detected (S104). For example, the imaging device 140 images the second electric field regions B and outputs images including particle species whose precipitation has been changed to negative. The detector 150 performs an image analysis on the output images to detect composite particles. As described above, the target substances contained in the composite particles are detected.

### Effects, etc

As described above, a detection method according to the present embodiment includes causing first dielectric particles 21 and second dielectric particles 22 to react with a sample 10 that contains a first target substance 11 and a second target substance 12, the first dielectric particles 21 being modified with first substances 21b having a property of specifically binding to the first target substance 11, the second dielectric particles 22 being modified with second substances 22b having a property of specifically binding to the second target substance 12 different from the first target substance 11 and having a different dielectrophoretic property from the first dielectric particles 21, by causing dielectrophoresis in the sample 10 after the reaction, separating a first composite particle 31 that is a first dielectric particle 21 to which the first target substance 11 is bound among the first dielectric particles 21 from the other first dielectric particle 21, and separating a second composite particle 32 that is a second dielectric particle 22 to which the second target substance 12 is bound among the second dielectric particles 22 from the other second dielectric particle 22, and detecting each of the first target substance 11 and the second target substance 12, the first target substance 11 being contained in the separated first composite particle 31, the second target substance 12 being contained in the separated second composite particle 32.

In such a detection method, a first composite particle 31 is formed by binding a first dielectric particle 21 and a first target substance 11 contained in the sample 10 and is separated from another first dielectric particle 21, a second composite particle 32 is formed by binding a second dielectric particle 22 and a second target substance 12 contained in the sample 10 and is separated from another second dielectric particle 22, and the first target substance 11 contained in the separated first composite particle 31 and the second target substance 12 contained in the separated second composite particle 32 can each be detected. Thus, the target substances can be appropriately and individually detected as the first composite particle 31 and the second composite particle 32.

For example, when the dielectrophoresis is caused, first dielectrophoresis and second dielectrophoresis may be caused in a time-division manner, the first dielectrophoresis being based on an alternating current voltage at a first frequency, the second dielectrophoresis being based on an alternating current voltage at a second frequency different from the first frequency.

According to this, the first dielectrophoresis is caused by applying the alternating current voltage at the first frequency, and dielectrophoretic movement of one particle species among the first composite particle 31, the second composite particle 32, the other first dielectric particle 21, and the other second dielectric particle 22 is changed from positive to negative or from negative to positive. Thereafter, the second dielectrophoresis is caused by applying the alternating current voltage at the second frequency, and dielectrophoretic movement of one particle species different from the particle species whose dielectrophoretic movement is changed in the case of the first dielectrophoresis is changed from positive to negative or from negative to positive, the one particle species being among the first composite particle 31, the second composite particle 32, the other first dielectric particle 21, and the other second dielectric particle 22. In this manner, the first target substance 11 contained in the separated first composite particle 31 and the second target substance 12 contained in the separated second composite particle 32 can each be detected. Thus, the target substances can be appropriately and individually detected as the first composite particle 31 and the second composite particle 32.

For example, the particle diameter of a base material portion (the first base material 21a) of the first dielectric particle 21 excluding the first substance 21b may be different from the particle diameter of a base material portion (the second base material 22a) of the second dielectric particle 22 excluding the second substance 22b.

According to this, a difference in dielectrophoretic properties between the first base material 21a and the second base material 22a can be caused on the basis of the particle diameter difference between the first base material 21a and the second base material 22a. That is, a difference in dielectrophoretic properties between the first dielectric particle 21 and the second dielectric particle 22 can be caused.

For example, when the dielectrophoresis is caused, the dielectrophoresis may be caused by generating a non-uniform electric field in the sample 10.

According to this, dielectrophoresis can be caused on the basis of the non-uniform electric field generated in the sample 10.

For example, each of the first substance 21b and the second substance 22b may be an antibody.

According to this, the first target substance 11 and the first dielectric particle 21 can be bound with high specificity by an antigen-antibody reaction, and the second target substance 12 and the second dielectric particle 22 can be bound with high specificity by an antigen-antibody reaction.

For example, a spectroscopic property of the base material portion (the first base material 21a) of the first dielectric particle 21 excluding the first substance 21b may be different from a spectroscopic property of the base material portion (the second base material 22a) of the second dielectric particle 22 excluding the second substance 22b.

According to this, the first dielectric particle 21 and the second dielectric particle 22 can be distinguished from each other also by the spectroscopic properties. For example, in a case where the dielectrophoretic properties of the first composite particle 31 and those of the second composite particle 32 match under conditions during dielectrophoresis, these particle species can be distinguished from each other using the spectroscopic properties. Thus, even under the above-described circumstances, the first target substance 11 contained in the separated first composite particle 31 and the second target substance 12 contained in the separated second composite particle 32 can each be detected. That is, even under the above-described circumstances, the target substances can be appropriately and individually detected as the first composite particle 31 and the second composite particle 32.

The detection apparatus 100 according to the present embodiment includes a reaction section that causes first dielectric particles 21 and second dielectric particles 22 to react with a sample 10 that contains a first target substance 11 and a second target substance 12, the first dielectric particles 21 being modified with first substances 21b having a property of specifically binding to the first target substance 11, the second dielectric particles 22 being modified with second substances 22b having a property of specifically binding to the second target substance 12 different from the first target substance 11 and having a different dielectrophoretic property from the first dielectric particles 21, a separator 110 that separates a first composite particle 31 that is a first dielectric particle 21 to which the first target substance 11 is bound among the first dielectric particles 21 from the other first dielectric particle 21 and separates a second composite particle 32 that is a second dielectric particle 22 to which the second target substance 12 is bound among the second dielectric particles 22 from the other second dielectric particle 22 by causing dielectrophoresis in the sample 10 after the reaction, and a detector 150 that detects each of the first target substance 11 and the second target substance 12, the first target substance 11 being contained in the separated first composite particle 31, the second target substance 12 being contained in the separated second composite particle 32.

The detection apparatus 100 as described above can provide the same effects as the detection method described above.

### Other Embodiments

The detection apparatuses and the detection methods according to one or more aspects of the present disclosure have been described above on the basis of the embodiment; however, the present disclosure is not limited to this embodiment. Various variations that one skilled in the art can conceive of and that are made without departing from the gist of the present disclosure may also be included within the scope of the one or more aspects of the present disclosure.

For example, in the above-described embodiment, a non-uniform electric field may be generated using a set of electrodes in which first projections of a first electrode face second recesses of a second electrode in the second direction.

The number of electrodes included in the set of electrodes is not limited to two and may be greater than or equal to three. A set of electrodes including three or more electrodes may be used, and a phase difference may be provided between AC voltages applied to adjacent electrodes. Such a set of electrodes may be referred to as Castellated electrodes.

By excluding the step of detecting from the detection method according to an aspect of the present disclosure, the method can also be realized as a separation method, and such a separation method is also included as an embodiment in the present disclosure.

That is, a separation method according to an aspect of the present disclosure includes causing first dielectric particles and second dielectric particles to react with a sample that contains a first target substance and a second target substance, the first dielectric particles being modified with first substances having a property of specifically binding to the first target substance, the second dielectric particles being modified with second substances having a property of specifically binding to the second target substance different from the first target substance and having a different dielectrophoretic property from the first dielectric particles, and by causing dielectrophoresis in the sample after the reaction, separating a first composite particle that is a first dielectric particle to which the first target substance is bound among the first dielectric particles from the other first dielectric particle, and separating a second composite particle that is a second dielectric particle to which the second target substance is bound among the second dielectric particles from the other second dielectric particle.

By excluding the detector from the detection apparatus according to an aspect of the present disclosure, the detection apparatus can also be realized as a separation apparatus, and such a separation apparatus is also included as an embodiment in the present disclosure.

That is, a separation apparatus according to an aspect of the present disclosure includes a reaction section that causes first dielectric particles and second dielectric particles to react with a sample that contains a first target substance and a second target substance, the first dielectric particles being modified with first substances having a property of specifically binding to the first target substance, the second dielectric particles being modified with second substances having a property of specifically binding to the second target substance different from the first target substance and having a different dielectrophoretic property from the first dielectric particles, and a separator that separates a first composite particle that is a first dielectric particle to which the first target substance is bound among the first dielectric particles from the other first dielectric particle and separates a second composite particle that is a second dielectric particle to which the second target substance is bound among the second dielectric particles from the other second dielectric particle by causing dielectrophoresis in the sample after the reaction.

A detection apparatus according to the present disclosure can be used to detect target substances, such as viruses that cause infectious diseases.

What is claimed is:

1. A detection method comprising:
causing first dielectric particles and second dielectric particles to react with a sample that contains a first target substance and a second target substance, the first dielectric particles being modified with first substances having a property of specifically binding to the first target substance, the second dielectric particles being modified with second substances having a property of specifically binding to the second target substance different from the first target substance and having a different dielectrophoretic property from the first dielectric particles;
by causing dielectrophoresis in the sample after the reaction, separating a first composite particle that is a first dielectric particle to which the first target substance is bound among the first dielectric particles from the other first dielectric particle, and separating a second composite particle that is a second dielectric particle to which the second target substance is bound among the second dielectric particles from the other second dielectric particle; and
detecting each of the first target substance and the second target substance, the first target substance being contained in the separated first composite particle, the second target substance being contained in the separated second composite particle.

2. The detection method according to claim 1,
wherein when the dielectrophoresis is caused, first dielectrophoresis and second dielectrophoresis are caused in a time-division manner, the first dielectrophoresis being based on an alternating current voltage at a first frequency, the second dielectrophoresis being based on an alternating current voltage at a second frequency different from the first frequency.

3. The detection method according to claim 1,
wherein a particle diameter of a base material portion of the first dielectric particle excluding the first substance is different from a particle diameter of a base material portion of the second dielectric particle excluding the second substance.

4. The detection method according to claim 1,
wherein when the dielectrophoresis is caused, the dielectrophoresis is caused by generating a non-uniform electric field in the sample.

5. The detection method according to claim 1,
wherein each of the first substance and the second substance is an antibody.

6. The detection method according to claim 1,
wherein a spectroscopic property of a base material portion of the first dielectric particle excluding the first substance is different from a spectroscopic property of a base material portion of the second dielectric particle excluding the second substance.

7. A detection apparatus comprising:
a reaction section that causes first dielectric particles and second dielectric particles to react with a sample that contains a first target substance and a second target substance, the first dielectric particles being modified with first substances having a property of specifically binding to the first target substance, the second dielectric particles being modified with second substances having a property of specifically binding to the second target substance different from the first target substance and having a different dielectrophoretic property from the first dielectric particles;
a separator that separates a first composite particle that is a first dielectric particle to which the first target substance is bound among the first dielectric particles from the other first dielectric particle and separates a second composite particle that is a second dielectric particle to which the second target substance is bound among the second dielectric particles from the other second dielectric particle by causing dielectrophoresis in the sample after the reaction; and
a detector that detects each of the first target substance and the second target substance, the first target substance being contained in the separated first composite particle, the second target substance being contained in the separated second composite particle.

8. A detection method comprising:
(a) accommodating a sample that contains first target substances and second target substances, first dielectric particles, and second dielectric particles in a first space, thereby the first space containing a first composite particle, a second composite particle, a third dielectric particle, and a fourth dielectric particle,
each of the first dielectric particles contains a first base material and a first substance, which modifies the first base material,
each of the second dielectric particles contains a second base material and a second substance, which modifies the second base material,
the first dielectric particles contain the third dielectric particle and a fifth dielectric particle,
the second dielectric particles contain the fourth dielectric particle and a sixth dielectric particle,
the first composite particle contains the fifth dielectric particle and a third target substance, which is contained in the first target substances, and the fifth dielectric particle and the third target substance are bound to each other via the first substance,
the second composite particle contains the sixth dielectric particle and a fourth target substance, which is contained in the second target substances, and the sixth dielectric particle and the fourth target substance are bound to each other via the second substance,
the third dielectric particle is not bound to any of the first target substances,
the third dielectric particle is not bound to any of the second target substances,
the fourth dielectric particle is not bound to any of the first target substances,
the fourth dielectric particle is not bound to any of the second target substances,
the first substance is capable of being directly bound to one of the first target substances,
the second substance is capable of being directly bound to one of the second target substances,
the first substance is incapable of being directly bound to any of the second target substances,
the second substance is incapable of being directly bound to any of the first target substances, and
a particle diameter of the first base material is larger than a particle diameter of each second base material; and
(b) applying, at different times, voltages having frequencies to a first region included in the first space, thereby the third dielectric particle, the fourth dielectric particle, the first composite particle, and the second composite particle being each detected, the frequencies being different from each other.
